(19) United States
(12) Patent Application Publication
(10) Pub. No.: US 2024/0269253 A1
(43) Pub. Date: Aug. 15, 2024

(54) DNA VACCINE FOR USE IN THE THERAPEUTIC AND/OR PROPHYLACTIC TREATMENT OF TUMOR DISEASES

(71) Applicant: UNIVERSITA' DEGLI STUDI DI TORINO, Torino (IT)

(72) Inventors: Francesco NOVELLI, Torino (IT); Paola CAPPELLO, Torino (IT); Claudia CURCIO, Piossasco (Torino) (IT); Silvia BRUGIAPAGLIA, Torino (IT)

(21) Appl. No.: 18/695,235

(22) PCT Filed: Sep. 27, 2022
(86) PCT No.: PCT/IB2022/059186

§ 371 Date: Mar. 25, 2024

(30) Foreign Application Priority Data
Sep. 28, 2021 (IT) 102021000024779

(51) Int. Cl. A61K 39/00; A61P 35/00; C12N 9/88; C12N 15/85

(52) U.S. Cl. CPC A61K 39/001154; A61P 35/00; C12N 9/88; C12N 15/85; A61K 2039/53; A61K 2039/572; A61K 2039/575; A61K 2039/852; C07K 2319/00; C12Y 402/01011

(57) ABSTRACT

A recombinant expression vector suitable for eliciting an immune response a subject having a tumor is provided. In addition to a promoter and additional transcription regulatory elements, the recombinant expression vector has a nucleotide sequence coding for an immunogenic synthetic peptide of SEQ ID NO:15.

The present invention falls within the field of immunotherapy as a prophylactic and/or therapeutic approach for the treatment of tumor diseases.

More specifically, the invention relates to a DNA vaccine for the prophylactic and/or therapeutic treatment of tumor diseases, preferably pancreatic ductal adenocarcinoma also referred to as “PDAC”.

PDAC is the most common of pancreatic tumors and the fourth leading cause of death in the United States and Europe but is expected to become the second leading cause by 2025. In fact, PDAC has a very poor prognosis with a median survival of 6 months and a 5-year survival rate from diagnosis of 8% [Siegel R L, Miller K D, Jemal A. Cancer Statistics, 2020. CA. Cancer J. Clin. 2020;70(1):7-30]. Surgery remains the curative treatment par excellence when an early diagnosis can be made, but this is only applicable to 10-20% of cases. The overall 5-year survival after pancreaticoduodenectomy is approximately 25-30% in node-negative tumors and 10% in node-positive cases, as locoregional or distant recurrence often occurs in the rest of the patients.

Despite considerable efforts in the field of medical oncology, the chemotherapy and radiotherapy treatments used increase survival only marginally.

In this context, there is an urgent need for innovative anti-tumor therapies which are capable of intervening more effectively on the pathogenetic mechanisms underlying the development of PDAC and other tumors, thereby enabling a significant improvement in patients' survival rate.

In recent decades, immunotherapy has played an important role in tumor treatment prospects, the development of which has been enabled by the identification of an ever-increasing number of tumor-associated antigens (TAA), which, by being expressed aberrantly in tumor cells, are a target against which to induce or restore a specific immune response capable of causing their death. Most of these TAAs are represented by self-proteins which can be recognized as such and thus trigger the regulatory and suppressive responses that physiologically maintain tissue homeostasis. This process, known as tolerance, is the main obstacle to the immune system's response to self-proteins, even if expressed aberrantly by tumor cells. One strategy to overcome the obstacle of tolerance to a self-protein is to change its sequence to make it more similar to non-self antigens against which a strong immune response can be triggered. In fact, in a context of immunological tolerance, self-proteins normally induce immune cells to produce soluble suppressive factors such as interleukin 10 (IL-10) and tumor growth factor beta (TGF-β) which inhibit the responses of antigen-activated effector T cells. One possible change is the removal of sequences that induce immune cells' suppressive responses.

Cancer vaccines are one of the most promising approaches in the field of cancer immunotherapy, on a par with - or even better than - monoclonal antibodies, as antibodies are not effective in many patients or cancer types.

Knowledge advances in the field of molecular and cellular biology have recently enabled the development of an alternative type of vaccine, based on the administration, rather than of the antigen as a protein capable of inducing a protective immune response, of the DNA sequence coding for the antigen protein inserted in a vector. In addition, RNA-based vaccines are known to have been developed very recently to counter the Sars-Cov-2 pandemic.

In the case of DNA vaccines, the recombinant DNA molecules used for immunization, once taken up by the target cells, cause the expression of the encoding sequence and the production of the corresponding protein, which is able to trigger a complete immune response. In fact, unlike conventional antigen vaccines which only induce humoral protection, DNA vaccines also allow the triggering of the major histocompatibility complex (MHC) class I pathway through intracellular antigen presentation, resulting in increased cell-mediated immunity. T lymphocytes are the only cells in the immune system capable of recognizing antigens through a specific membrane receptor (TCR), which binds peptides derived from proteins housed in a pocket of the MHC molecules—which are expressed on the cell surface and in humans are called human leukocyte antigens (HLA)—allowing T lymphocytes to recognize antigens deriving from proteins located within the cell. Peptides of intracellular or endogenous origin are presented within Class I MHCs to CD8⁺ cytotoxic T lymphocytes. Antigens of exogenous origin or derived from the phagocytosis of proteins or cells are presented within Class II MHCs to CD4⁺ T helper lymphocytes. The latter are essential to activate cytotoxic lymphocytes and B lymphocytes and thus trigger an inflammatory and anticancer antibody response.

A further advantage of nucleic acid (DNA or RNA) vaccines is the synthesis of the immunogenic protein directly in the host organism, allowing the cell to be provided with the genetic information required for in vivo production of complex antigens, which would otherwise be difficult to isolate or synthesize in vitro, and at the same time guaranteeing the production of proteins characterized by the same conformation.

Among cancer-associated antigens in humans, CA19.9 Lewis blood-type sialylated antigen is currently considered the most important diagnostic and prognostic serological marker, despite the significant amount of evidence indicating its reduced specificity.

In order to identify more reliable tumor markers, in recent years studies have been performed on blood and tissues of cancer patients, thanks to which, using techniques analysing large-scale RNA or protein expression, the expression levels of a large number of human proteins could be monitored in relation to the onset and progression of cancer and its prognostic pattern.

The research described in Tomaino B, Cappello P, Capello M, et al. Circulating autoantibodies to phosphorylated a-enolase are a hallmark of pancreatic cancer. J Proteome Res. 2011;10(1): 105-112 on the serum-proteome profiles of a large cohort of PDAC patients and their controls revealed a specific association between this tumor and the increase in pancreatic levels of the glycolitic enzyme alpha-enolase (“ENO1” or “ENOA”) and, in particular, of its isoforms phosphorylated on serine in position 419. In addition, circulating autoantibodies against the phosphorylated epitopes of the ENOA1-2 isoforms were found in 62% of patient sera, unlike what was found in the control group in which the aforementioned immunoreactivity was only present in 4% of samples. To further support the clinical value of these findings, the authors showed that the antibody response to phosphorylated alpha-enolase isoforms correlates in most cases with a more favorable prognosis and a significant increase in survival estimate. Furthermore, two parallel studies demonstrated the presence of T lymphocytes capable of specifically recognizing the ENO1 protein and to become activated. These cells have been isolated from both PDAC patients' blood, where they correlate with the presence of circulating antibodies against ENO1 itself [Cappello P. Tomaino B. Chiarle R, et al. An Integrated Humoral and Cellular Response Is Elicited in Pancreatic Cancer by α-Enolase, a Novel Pancreatic Ductal Adenocarcinoma-Associated Antigen. Int. J. Cancer. 2009;125(3):639-648], and from biopsies [Amedei A, Niccolai E, Benagiano M, et al. Ex vivo analysis of pancreatic cancer-infiltrating T lymphocytes reveals that ENO-specific Tregs accumulate in tumor tissue and inhibit Th1/Th17 effector cell functions. Cancer Immunol Immunother. 2013;62(7): 1249-1260].

The specific linkage of the alpha-enolase antigen with PDAC makes this protein an ideal candidate for the development of a prognostic marker for PDAC. Patent WO2011/030302 A1 describes the use of the human alpha-enolase phosphorylated isoform as a biomarker for the diagnosis of PDAC, together with peptides derived therefrom containing the phosphorylation site and with antibodies capable of specifically binding the phosphorylated epitope.

It is also known that the ENO1 antigen is overexpressed on the surface of a myriad of cancer cell types other than PDAC and correlates with disease progression, making it an excellent diagnostic and prognostic tumor marker. There are also several works showing how strategies for targeting ENO1 can be effective in many types of cancer, such as, but not limited to, lung cancer, cervical cancer, gastric cancer, hepatocellular carcinoma and breast cancer. See e.g. Huang CK, Sun Y, Lv L, et al. ENO1 and Cancer. Molecular therapy oncolytics, 2022;24:288-298; Cappello P. Principe M, Bulfamante S, et al. Front Biosci (Landmark Ed). 2017;22(5):944-959; Almaguel FA, Sanchez TW, Ortiz-Hernandez et al. Front Genet. 2021;11:614726.

International application WO2007/072219 discloses the use of the full-length ENO1 sequence for both diagnostic and therapeutic applications in the oncology field. The object of the above patent application is to target ENO1 contained in tumor cells to inhibit the growth and survival thereof.

Further research has been carried out to use the ENO1 antigen in the therapeutic field in order to develop approaches that, either alternatively or in combination with conventional strategies, enable effective action against tumor cells, thereby slowing down the progression of the neoplastic disease.

International application WO2016/170139 describes a short ENO1 peptide of only 11 amino acids as a therapeutic strategy in the treatment of tumors.

A DNA vaccine based on a nucleotide sequence coding for full-length human ENO1 (pVAXENO1) is described in Cappello P, Rolla S, Chiarle R, et al. Vaccination with ENO1 DNA prolongs survival of genetically engineered mice with pancreatic cancer. Gastroenterology. 2013;144(5):1098-1106, where the authors show that 3 or 4 immunizations with the vector expressing the full-length ENO1 protein prolong the life expectancy by almost 30% in mice spontaneously developing PDAC.

The human ENO1 protein sequence is available in the UniProt database under accession number P06733 (SEQ ID NO:39). It is a fairly large protein, having a length of 434 amino acids and a mass of 47169 Da.

However, it is known that proteins of a certain length may contain non-immunogenic amino acid regions that reduce the extent and selectivity of the immune response, stimulating the activation of suppressor lymphocytes that switch off CD4 and CD8 lymphocytes, and the antibody response.

In order to overcome this drawback, the present inventors analysed the sequence of the native human ENO1 protein (SEQ ID NO:39) and identified the regions that are actually immunogenic and therefore suitable to be used in a nucleic acid-based anticancer vaccine.

The inventors found that these immunogenic regions of human ENO1 are located in the N- terminal domain of the protein and consist of ENO1 sequences designated as SEQ ID NO:1, SEQ ID NO:2, SEQ ID NO:4, SEQ ID NO:5, SEQ ID NO:8 and SEQ ID NO:9 shown below in Table 1.

It will be noted that some of the above sequences partially overlap, as they have been selected from a library of 14 ENO1 peptides of 50 amino acids each (except the last peptide of 44 amino acids) with a consecutive overlap of 20 amino acids, which together cover the full-length human ENO1 amino acid sequence, from the N-terminal end of the protein to the C-terminal end.

By fusing two or more of the above ENO1 immunogenic regions (where “fusing” is understood as the combination of the respective amino acid sequences without duplication of the overlapping regions), and excluding combinations giving rise to peptides corresponding to fragments of the native human ENO1 protein, the inventors designed immunogenic synthetic peptides that do not contain non-immunogenic regions and are therefore capable of eliciting a particularly strong anticancer immune response when administered to a patient, either as such or as nucleic acid (e.g., DNA) constructs coding therefor. These immunogenic synthetic peptides are also characterized by the fact that they do not correspond to, i.e., they are different from, fragments of the native human ENO1 protein and are therefore not naturally occurring.

The immunogenic synthetic peptides designed by the inventors also have the feature of being non-self and of not being subjected to immunological tolerance, as they are deprived of the ability to induce suppressive responses. In addition, unlike the ENO1 peptides described in the state of the art, and in particular unlike the peptide of SEQ ID NO:47 described in WO2016/170139, which can be only presented by individuals with HLA*A02 and HLA*A24 alleles, the immunogenic synthetic peptides designed by the inventors are recognized virtually by all HLA haplotypes of both class I and class II, thus avoiding the need for prior HLA typing of the patient.

Therefore, a first aspect of the invention is a recombinant expression vector comprising a recombinant nucleotide sequence coding for an immunogenic synthetic peptide resulting from the fusion of two or more of the amino acid sequences SEQ ID NO:1, SEQ ID NO:2, SEQ ID NO:4, SEQ ID NO:5, SEQ ID NO:8, or SEQ ID NO:9 of the human ENO1 protein, said recombinant nucleotide sequence being operatively linked to a promoter sequence and any additional transcription regulatory elements, excluding immunogenic synthetic peptides corresponding to fragments of the native human ENO1 protein.

Preferably, the recombinant expression vector of the invention codes for an immunogenic synthetic peptide selected from the group consisting of SEQ ID NOs: 15-38. The preferred sequences SEQ ID NOs: 15-38 are shown in Table 4 below.

A particularly preferred embodiment is a recombinant expression vector encoding the peptide having the amino acid sequence SEQ ID NO: 15, resulting from the fusion of all the human ENO1 protein immunogenic regions identified by the inventors.

A second aspect of the invention is an immunogenic synthetic peptide resulting from the fusion of two or more of the amino acid sequences SEQ ID NO:1, SEQ ID NO:2, SEQ ID NO:4, SEQ ID NO:5, SEQ ID NO:8, or SEQ ID NO:9 of the human ENO1 protein, excluding peptides that correspond to fragments of the native human ENO1 protein. In a preferred embodiment, the immunogenic synthetic peptide of the invention is selected from the group consisting of SEQ ID NOs: 15-38; even more preferably, the immunogenic synthetic peptide of the invention is SEQ ID NO:15.

Further preferred embodiments of the invention form the object of the remaining dependent and independent claims, the content of which forms an integral part of the present specification.

As mentioned above, the term “immunogenic” indicates the ability to elicit an immune response in the target organism. Therefore, the immunogenic synthetic peptides of the invention, as well as the recombinant expression vectors encoding them, are capable of eliciting an immune response against tumor cells, so they are suitable to be used both as prophylactic vaccines and as therapeutic vaccines against various types of tumors.

The protection conferred by the recombinant expression vector of the invention is achieved by its inoculation in a patient, where it is translated into the immunogenic peptide encoded by it, which has the property of being highly immunoreactive and thus capable of activating the patient's immune system.

As will be illustrated in greater detail in the following experimental part, a significant advantage of this immunotherapeutic approach is the induction of a complete and integrated immune response consisting of a humoral component, associated with a considerable increase in the serum level of anti-ENO1 specific IgG immunoglobulins, and at the same time of a cell-mediated component, represented by the activation of ENO1-specific T lymphocytes.

It is known that T responses can also be induced by modified ENO1 peptides, as suggested in the paper by Capello M, Caorsi C, Bogantes Hernadez P J, et al. Phosphorylated alpha-enolase induces autoantibodies in HLA-DR8 pancreatic cancer patients and triggers HLA-DR8 restricted T cell activation. Immunology Letters. 2015;167(1):11-16 and in WO2017/013425. WO2017/013425 describes citrullinated ENO1 peptides for both prophylactic and therapeutic use against tumors. However, anti-citrullinated ENO1 antibodies are also known to be associated with the development of rheumatoid arthritis [Kinloch A, Tatzer V, Wait R et al. Identification of citrullinated alpha-enolase as a candidate autoantigen in rheumatoid arthritis. Arthritis Res Ther. 2005;7(6):R1421-9; Lundberg K, Kinloch A, Fisher B A, et al. Autoantibodies to citrullinated alpha-enolase peptide 1 are specific rheumatoid arthritis and cross-react with bacterial enolase. Arthritis Reum. 2008;58(10):3009-19; Mahdi H, Fisher B A, Kallberg H et al. Specific interaction between genotype, smoking and autoimmunity to citrullinated alpha-enolase in the etiology of rheumatoid arthritis. Nat Genet. 2009;41(12): 1319-24].

The state of the art therefore suggests that the use of citrullinated peptides for therapeutic purposes could lead to an unwanted autoimmune response. One advantage of the present invention is that the immunogenic synthetic peptides designed by the inventors do not involve post-translational modifications, so the risk of adverse immune reactions is extremely limited.

A third aspect of the invention is the use of the recombinant expression vector as defined above, or of the immunogenic synthetic peptide as defined above, in the prophylactic or therapeutic treatment of a tumor in a human or animal subject. The animal is preferably a mammal, even more preferably it is selected from a dog, cat, pig, cow, and horse. According to a preferred embodiment, the tumor is pancreatic ductal adenocarcinoma.

As stated above, the recombinant expression vector of the invention comprises a promoter sequence and any additional transcription regulatory sequences. A transcription regulatory sequence is for example a polyadenylation signal sequence.

For example, the vector may be a bacterial plasmid in which the recombinant encoding nucleotide sequence is under the control of a strong viral promoter. Techniques for preparing vectors containing the aforementioned regulatory elements and any additional elements, such as one or more cloning sites, one or more enhancer sequences, a sequence encoding a signal peptide, one or more marker genes such as for example antibiotic resistance genes, and/or one or more synthetic introns, fall within the knowledge and skills of those of ordinary skill in the art.

In a preferred embodiment, the recombinant expression vector is unable to replicate in a mammalian cell. In order to suppress the ability to replicate in target cells, a number of measures may be taken, including, but not limited to, the use of vectors containing one or more prokaryotic origins of replication.

The inventors have verified that the pVAX1 plasmid is particularly suitable for use as a vector within the scope of the invention. The pVAX1 plasmid contains a pUC origin of replication, a Cytomegalovirus (CMV) viral promoter, and restriction sequences for the enzymes NotI and XbaI. However, other vectors known per se to be suitable for use in DNA or RNA vaccines can be used and readily selected by those of ordinary skill in the art. Examples include, but are not limited to, the plasmid vector pVAC, pCDNA3, or viral vectors such as for example adenoviral or adeno-associated viral vectors.

The inability of the recombinant vector to replicate in the host cells and thus to integrate into their genome gives the vaccine a high safety profile.

Preferably, the recombinant expression vector of the invention is provided in the form of a pharmaceutical composition comprising, in addition to the recombinant expression vector, pharmaceutically acceptable excipients, carriers and/or diluents.

In addition, the pharmaceutical composition optionally comprises one or more adjuvants capable of enhancing the effectiveness of the immune response elicited by the recombinant vector on the effector, antibody, and cellular systems.

Adjuvants are a heterogeneous family of compounds that differ in their chemical structure and mechanism of action, including mineral substances, oil emulsions, and bacterial derivatives. Substances suitable for use as adjuvants in the pharmaceutical composition of the invention include, but are not limited to, Toll-like receptor agonists such as CpG sequences and the compound Imiquimod, the inflammatory mediator High-Mobility Group Protein B1 (HMGB1), iNKT lymphocyte synthetic agonists, and γδT-lymphocyte agonists.

Optional additional components of the pharmaceutical composition of the invention are, for example, substances with stabilizing and/or preservative functions.

In a preferred embodiment, the pharmaceutical composition of the invention is in a formulation suitable for oral, nasal, intradermal, subcutaneous, or intramuscular administration.

The DNA of the pharmaceutical composition of the invention may be in the form of a suspension in an appropriate medium, such as for example a saline buffer, therefore in a form particularly suitable for parenteral administration. Alternatively, the DNA molecules of the pharmaceutical composition of the invention can be delivered to the target tissue encapsulated by liposomes or adsorbed on microparticles consisting of polylactide-co-glycolide (PLG), i.e., a biocompatible, biodegradable polymer capable of preventing the degradation of the vaccine DNA.

Recently, in order to increase the immunogenicity of DNA vaccines, various strategies have been implemented which, when combined with the traditional methods of vaccine administration, allow the absorption of a significantly higher number of vaccine DNA molecules. Examples include the electroporation technology, which is applied to target tissue cells at the vaccine inoculation site, usually after intramuscular or intradermal injection, resulting in the opening of cell membranes and facilitating the entry of DNA molecules. In the case of intradermal vaccinations, remarkable results have been achieved by using special devices designated as “gene guns” that allow DNA molecules adhered to gold microspheres to be introduced at high pressure through the skin.

The pharmaceutical composition of the invention can be administered as a single dose or as repeated doses at predetermined time intervals.

The selection of the appropriate type of vaccine formulation, method of administration and dosage in order to achieve high protective efficacy falls within the skills of those of ordinary skill in the art. The selection of the carrier and any pharmaceutical excipients also falls within the skills of those of ordinary skill in the art.

Finally, the recombinant expression vector of the invention is suitable to be administered as a combined therapy with a second active substance known per se to be effective in the therapeutic treatment of cancer, such as a chemotherapeutic agent and/or an immunomodulating agent.

Therefore, a further aspect of the invention is a combined preparation comprising a recombinant expression vector of the invention and at least one chemotherapeutic agent and/or at least one immunomodulating agent for simultaneous, separate, or sequential use in the prophylactic or therapeutic treatment of a tumor in a subject. Per se known immunomodulators suitable for use in combination therapy with the recombinant vector of the present invention include, but are not limited to, suppressive cytokine inhibitors such as antibodies or Sh RNA molecules against interleukin 10, drugs inhibiting the suppressive activity of Treg lymphocytes, e.g., cyclophosphamide and anti-IL-2Ra (CD25) antibodies, costimulatory molecules such as the B7-IgG fusion molecule, MSDC cell inhibitors including inhibitors specific for the phosphodiesterase-5 enzyme such as the compounds Sildenafil, Tadalafil, and Vardenafil, and anti-CTLA4 monoclonal antibodies.

### BRIEF DESCRIPTION OF THE FIGURES

FIG. 1 shows the results of the in silico analyses carried out with the NetMHC-4.0 and NetMHCII-2.3 bioinformatics programs as described in Example 1. These results are graphically represented as heatmaps. These heatmaps report the prediction values as %-Rank. The dark blue squares indicate a strong binding affinity between the peptide and the HLA allele, whereas the white squares indicate no or almost no binding affinity. Each column corresponds to one of the 14 ENO1 peptides and each row corresponds to an HLA-A (panel A), HLA-B (panel B) and HLA-DRB1 (panel C) allele. Panels A and B represent the prediction map for the HLA-A and HLA-B loci obtained with NetMHC-4.0 by setting the threshold at 1%-Rank. Panel C represents the prediction map obtained from NetMHCII-2.3 by setting the threshold at 2%-Rank.

FIG. 2 shows three pie charts representing the gene frequency percentages of the HLA-A, HLA-B and HLA-DRB1 loci present in the tested donor cohort compared to the totality of gene frequencies present in the Italian population [Amoroso A, Ferrero NM, Rendine S et al. Le Caratteristiche HLA Della Popolazione Italiana: Analisi Di 370.000 Volontari Iscritti All'IBMDR. Analysis. 2010; 1-2, 23-102].

FIG. 3 shows the results of experiments carried out on a cohort of healthy donors related to the proliferative response induced by full-length recombinant ENO1 (rENO1) and by the 14 ENO1 peptides in Table 1. Panel A describes the proliferative capacity, referred to as the Stimulation Index (SI), of the donor cohort T lymphocytes stimulated with the 14 ENO1 peptides and rENO1. Each blue circle represents a donor, and the horizontal line of each column represents the mean. Panel B shows the typing and SI for each donor, highlighted in blue when ≥ 2. Panel C shows the “immunological tone”, expressed as the ratio between IFN-γ and IL-10 production. Values greater than 1 indicate an effector phenotype, shifted towards IFN-γ production, whereas values less than 1 indicate a suppressor phenotype, shifted towards IL-10 production. Statistical significance is shown in each graph.

FIG. 4 refers to experiments for the validation of the ENO1 immunogenic peptides in a cohort of PDAC patients. Panel A shows the proliferation, measured as the SI, of T lymphocytes stimulated with the 6 ENO1 immunogenic peptides and rENO1. Each blue circle represents a patient, and the horizontal line of each column represents the mean. Panel B shows the typing, SI, and value of the IFN-γ/IL-10 ratio, i.e., an index of the immunological tone, for each patient. The effector response (IFN-γ/IL-10>1) has been highlighted with a red gradation, whereas the suppressor response (IFN-γ/IL-10<1) has been highlighted with a blue gradation. T lymphocytes from patients stimulated with the selected peptides show a prevalent effector response, unlike those stimulated with rENO1 that show a prevalent suppressor response. Statistical significance is shown in each graph.

FIG. 5, Panel A, shows the sequence of the 6 ENO1 immunogenic peptides expressed as fusion proteins according to one embodiment of the invention, plus the two restriction sequences (underlined). Panel B shows the map of the vector obtained by inserting the sequence coding for SEQ ID NO: 15 inside the pVAX vector (pVAXENO3PEP).

FIG. 6 shows the effectiveness of vaccination with pVAXENO3PEP compared to that with the full-length ENO1 sequence in mice genetically engineered to spontaneously develop pancreatic cancer (GEM). pVAXENO3PEP vaccination reduces the tumor area in the pancreas (panel A) by inducing a strong ENO1-specific antibody response (panel B), increasing the number of IFN-γ-secreting T lymphocytes (panel C), and recruiting CD8+ and CD4+ T lymphocytes at the tumor site (panel D).

The experimental section that follows is provided for illustration purposes only and does not limit the scope of the invention as defined in the appended claims.

### EXPERIMENTAL SECTION

### Materials and Methods

### Preparation of the Biological Sample

Peripheral blood mononuclear cells (PBMC) were obtained from volunteers enrolled in the blood donor register at the Blood Bank and Immunohematology of the Città della Salute e della Scienza in Turin and from PDAC patients enrolled in the ENOAPA project, approved by the ethics committee of the Azienda Ospedaliera Citta della Salute e della Scienza in Turin.

The PBMCs were isolated from venous blood by fractionation of whole blood by density gradient centrifugation using HiSep medium (Himedia Cell Culture, Einhausen, Germany). The isolated PBMCs were frozen in RPMI medium (EuroClone spa, Milan, Italy) and 10% dimethylsulfoxide (DMSO, Sigma-Aldrich, Milan, Italy) and stored in liquid nitrogen.

### HLA Typing

All healthy donors and PDAC patients were typed for class I (A and B loci) and class II (DRB1) HLA alleles. HLA typing was performed on genomic DNA extracted from whole blood samples using high resolution Luminex technology.

### In Silico Prediction of Epitopes

The NetMHC-4.0 method (DTU Health Tech, Lyngby, Denmark) identifies 9-amino acid epitopes capable of binding HLA class I supertypes with greater affinity. The NetMHCII-2.3 method (DTU Health Tech) identifies 15-amino acid epitopes capable of binding HLA-DRB1 allele with greater affinity. Prediction values were given as %-Rank vs. a group of 1,000,000 random, naturally occurring peptides. The threshold used to define a high-affinity peptide is 1%-Rank for the NetMHC-4.0 analysis, and 2%-Rank for the NetMHCII-2.3 analysis.

### ENO1 Peptide Library

Peptides were synthesized by PEPperPRINT GmbH (Heidelberg, Germany). All peptides have a purity higher than 95% as indicated by high-performance liquid chromatography analysis. Lyophilized peptides were diluted in molecular biology grade water at a final concentration of 1 mg/ml. Aliquots were stored at -20° ° C. The library consists of 14 peptides of 50 amino acids each, except the last which has 44 amino acids, with a consecutive overlap of 20 amino acids, thus covering the full-length ENO1 amino acid sequence, starting from the N-terminal end of the protein to the C-terminal end (Table 1).

TABLE 1
Amino acid sequences from the ENO1 peptide library
used in the study

| | a.a. | |
|---|---|---|
| SEQ | posi- | |
| ID | tions | Amino acid sequence |
| 1 | 1-50 | MSILKIHAREIFDSRGNPTVEVDLFTSKGLFRAAVPS |
| | | GASTGIYEALELR |
| 2 | 31-80 | FRAAVPSGASTGIYEALELRDNDKTRYMGKGVSKAVE |
| | | HINKTIAPALVSK |
| 3 | 61-110 | GVSKAVEHINKTIAPALVSKKLNVTEQEKIDKLMIEM |
| | | DGTENKSKFGANA |
| 4 | 91-140 | DKLMIEMDGTENKSKFGANAILGVSLAVCKAGAVEKG |
| | | VPLYRHIADLAGN |
| 5 | 121-170 | AGAVEKGVPLYRHIADLAGNSEVILPVPAFNVINGGS |
| | | HAGNKLAMQEFMI |
| 6 | 151-200 | NVINGGSHAGNKLAMQEFMILPVGAANFREAMRIGAE |
| | | VYHNLKNVIKEKY |
| 7 | 181-230 | AMRIGAEVYHNLKNVIKEKYGKDATNVGDEGGFAPNI |
| | | LENKEGLELLKTA |
| 8 | 211-260 | GGFAPNILENKEGLELLKTAIGKAGYTDKVVIGMDVA |
| | | ASEFFRSGKYDLD |
| 9 | 241-290 | VIGMDVAASEFFRSGKYDLDFKSPDDPSRYISPDQLA |
| | | DLYKSFIKDYPVV |
| 10 | 271-320 | ISPDQLADLYKSFIKDYPVVSIEDPFDQDDWGAWQKF |
| | | TASAGIQVVGDDL |
| 11 | 301-350 | WGAWQKFTASAGIQVVGDDLTVTNPKRIAKAVNEKSC |
| | | NCLLLKVNQIGSV |
| 12 | 331-380 | AVNEKSCNCLLLKVNQIGSVTESLQACKLAQANGWGV |
| | | MVSHRSGETEDTF |
| 13 | 361-410 | QANGWGVMVSHRSGETEDTFIADLVVGLCTGQIKTGA |
| | | PCRSERLAKYNQL |
| 14 | 391-434 | GQIKTGAPCRSERLAKYNQLLRIEEELGSKAKFAGRN |
| | | FRNPLAK |

### In Vitro Assays on PBMCs

Donor PBMCs were seeded at a density of 5x106 per well in serum-free TexMACS medium (Miltenyi Biotec, Bologna, Italy) in 6-well plates and stimulated with the full-length rENO1 sequence at a concentration of 10 μg/ml (Sigma-Aldrich). After 3 days of culture, human recombinant IL-2 (rIL-2, Peprotech, Hamburg, Germany) was added at a concentration of 10 U/ml. After one week, T lymphocytes stimulated and expanded in the presence of rENO1 were added with, as the antigen-presenting cells, autologous PBMCs irradiated (3000 rad) in a 1:1 ratio, previously loaded with each of the 14 ENO1 peptides set out in Table 1.

PBMCs from PDAC patients were seeded at a concentration of 0.1×10⁶ per well in serum-free TexMACS medium (Miltenyi Biotec) in a 96-well plate and stimulated with 10 μg/ml of the individual peptides (SEQ ID NOs: 1, 2, 4, 5, 8, 9) or rENO1. After 3 days of culture, rIL-2 (Peprotech) was added at a concentration of 10 U/ml.

The proliferation and production of IFN-γ e IL-10 cytokines by donor and patient T lymphocytes was assessed 5 days after stimulation. Proliferation was measured by incorporation of bromodeoxyuridine (BrdU) as Time-Resolved Fluorescence (TRF) (PerkinElmer, Milan, Italy). The stimulation index of T-lymphocyte proliferation was calculated with the following formula: TRF from PBMCs grown in the presence of peptides or rENO1/TRF from PBMCs grown in the presence of stimulus-free medium alone. A stimulation index above 2 is considered as a positive value. IFN-γ and IL-10 production was measured by ELISA test (BioLegend, Campoverde, Milan, Italy) following the protocol supplied by the manufacturer. The IFN-γ to IL-10 concentration ratio—designated as the immunological tone—was used to evaluate the donor and patient responses to the individual peptide or the full-length protein as effector or suppressor responses. In fact, a prevalent production of IFN-γ is known to cause an anti-tumor immune response, designated as an “effector response”, whereas a prevalent production of IL-10 results in inhibition of the anti-tumor response, designated as a “suppressor response”.

### In Vivo Immunization

GEM mice were anesthetized with Zoletil (Rompun) and Xylazine and subsequently inoculated into the femoral muscle with 50 μg of either the empty plasmid, or the plasmid coding for ENO1 or SEQ ID 15 in 40 μl of sterile water with 0.9% NaCl. Immediately afterwards, two 25-ms 150-V pulses were applied 300 ms apart.

### Anti-ENO1 Antibody Assay (ELISA) and T-lymphocyte Activation Analysis (EliSPOT)

The recombinant human ENO1 protein at a concentration of 2 ug/ml was adhered and incubated overnight at 4° C. Mouse serum samples were diluted 1:50 in PBS containing 1% Bovine Serum Albumin (BSA) and 0.05% Tween-20. After 2 hours at room temperature, the plates were washed 8 times with PBS containing 0.05% Tween 20. A Horse Radish Peroxidase (HRP)-conjugated anti-mouse IgG (GE Healthcare) diluted 1:2000 was then added for one hour at room temperature. After 8 washes as described above, tetramethylbenzidine (TMB) (Tebu Bio, Magenta, Italy) was added for 20 minutes, after which the reaction was stopped with 2N HCI and the plates were read at 450 nm. Positivity was defined as the difference in the absorbance read in the wells in which the sera were incubated on the adhered recombinant ENO1 minus the absorbance of the empty wells in which the sera were incubated without the protein.

IFN-γ production from splenocytes stimulated ex vivo with ENO1 was assessed with a murine IFN-γ ELISPOT kit (Immunospot; CTL Europe, Bonn, Germany) following the manufacturer's instructions. Images of the wells were acquired, and the spots were quantified using a microplate reader, together with a computer-assisted image analysis system (Immunospot).

### Histology and Immunohistochemistry

The pancreases of the GEM mice were fixed in formalin and embedded in paraffin. The tissues were then stained with hematoxylin and eosin, or with antibodies specific for murine CD4 and CD8. For immunohistochemical staining, peroxidase activity was inhibited by a 3% aqueous hydrogen peroxide solution for 10 minutes. Samples were pre-treated using EDTA buffer at pH9 and incubated with anti-CD4 antibody (Abcam, Cambridge, UK, diluted 1:1000) or anti-CD8 antibody (Abcam, diluted 1:200), for 30 minutes at room temperature. This was followed by incubation with rabbit EnVision antibody (Dako) for 30 minutes at room temperature and then with diaminobenzidine tetrahydrochloride (Dako, Milan, Italy) for 5 minutes. The tissues were scanned (NanoZomer, Hamamatsu, Shizuoka, Japan) and the percentage of positive tumor area and cells in the tumor area was analysed using the QuPath program (University of Edinburgh).

Statistical analysis

Statistical analysis of the data obtained was performed using the GraphPad program (version 8, San Diego, CA) and the ANOVA test. Statistically significant groups are shown in the respective graphs.

### RESULTS

### In Silico Prediction of the Epitopes Most Recognized by T-lymphocytes From Healthy Donors in the Full-Length ENO1 Sequence

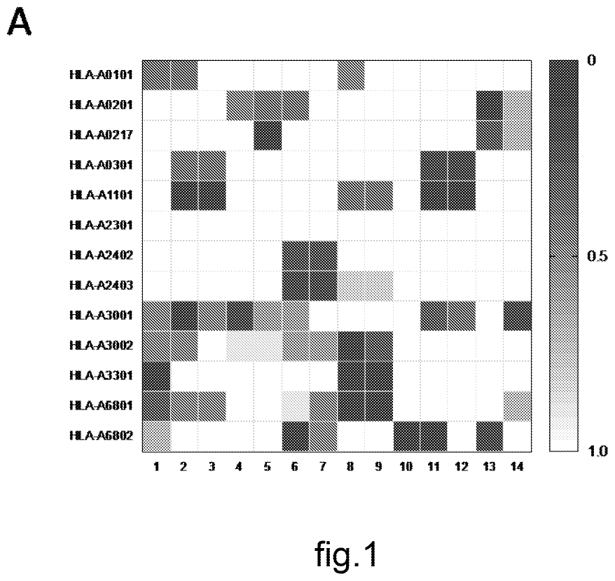

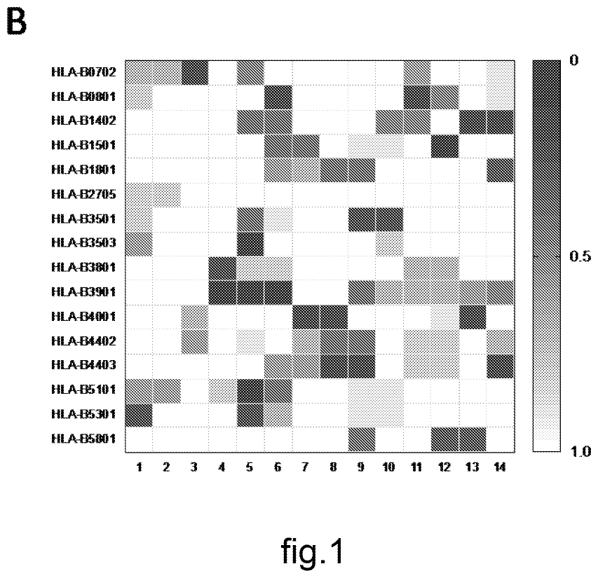

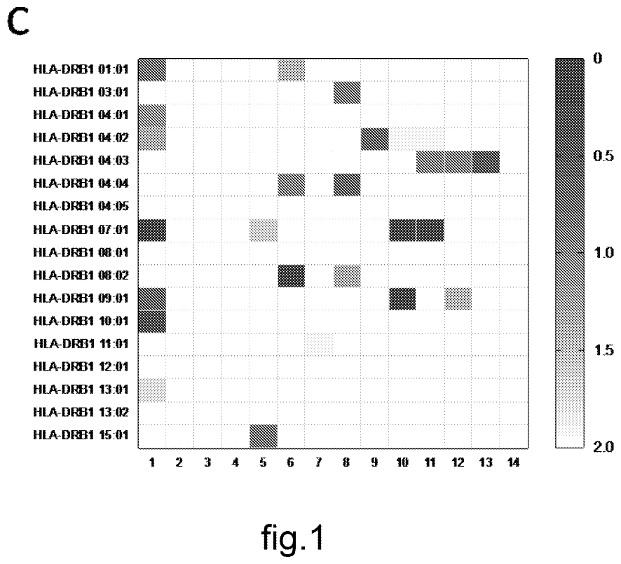

Two bioinformatics programs, NetMHC-4.0 and NetMHCII-2.3, were used to identify ENO1 epitopes binding with greater affinity class I and class II HLA molecules, respectively. As is known, the prediction of the binding specificity of class II HLA alleles is less accurate than that of class I due to increased variability in both the length and composition of the bound amino acid sequence, i.e., 9 amino acids for class I HLA alleles and 15 amino acids for class II HLA alleles. As shown in FIG. 1, the peptides of SEQ ID NOs: 1, 2, 4, 5, 8, 9 are among those predicted by the program that are capable of binding most of the HLA-A and HLA-B alleles (panel A and B, respectively). The same analysis, considering the class II DRB1 allele, in the presentation highlights the same peptides as potentially linked by most of the alleles (FIG. 1C).

### Assessment of the Proliferative Index and Cytokine Response of T lymphocytes in a Cohort of Healthy Donors Representative of the Italian Population

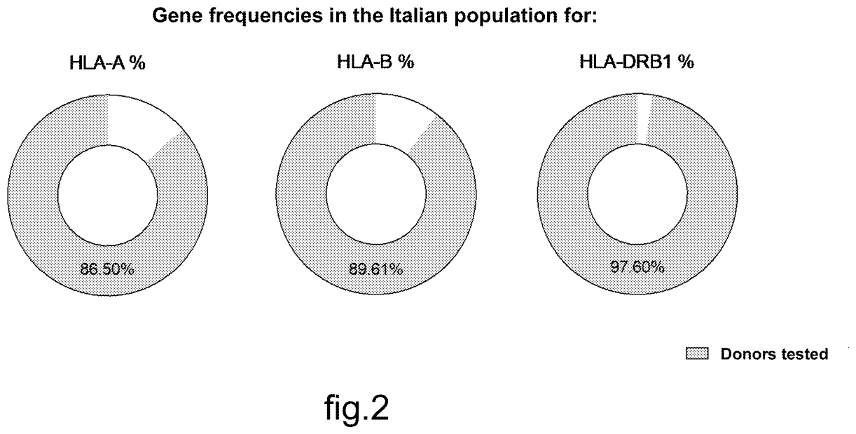

In vitro immunoassays were then used to validate the in silico prediction and complete the identification of the most immunogenic epitopes. A cohort of 17 healthy donors (Table 2) representing the most frequent HLA alleles in the Italian population was selected. In fact, as can be seen from the graph in FIG. 2, based on the distribution of the gene frequencies in the Italian population [Amoroso A, Ferrero NM, Rendine S et al. Le Caratteristiche HLA Della Popolazione Italiana: Analisi Di 370.000 Volontari Iscritti All'IBMDR. Analysis. 2010; 1-2, 23-102], the donor HLA haplotypes used for the study cover 86.50% of the frequencies of the HLA class I locus A, approximately 90% of the frequencies of the HLA class I locus B, and almost all the frequencies of the HLA-DRB1.

TABLE 2
Typing for the HLA-A, HLA-B and HLA-DRB1 loci
of each donor of the cohort used in the study

| | Typing | | |
|---|---|---|---|
| Donors | HLA-A* | HLA-B* | HLA-DRB1* |

| | | | | | | |
|---|---|---|---|---|---|---|
| 1 | 01:01 | 03:02 | 35:03 | 58:01 | 03:01 | 11:01 |
| 2 | 02:01 | 68:01 | 35:03 | 51:01 | 07:01 | 11:01 |
| 3 | 02:01 | 24:02 | 07:02 | 15:01 | 11:03 | 15:01 |
| 4 | 02:17 | 03:01 | 18:01 | 51:01 | 09:01 | 11:01 |
| 5 | 02:01 | 11:01 | 18:01 | 35:01 | 04:02 | 11:04 |

| | | | | | |
|---|---|---|---|---|---|
| 6 | 02:01 | 13:02 | 18:01 | 07:01 | 14:01 |

| | | | | | | |
|---|---|---|---|---|---|---|
| 7 | 23:01 | 24:03 | 38:01 | 44:03 | 07:01 | 14:01 |
| 8 | 23:01 | 24:02 | 14:02 | 18:01 | 11:04 | 14:01 |
| 9 | 30:04 | 33:01 | 14:02 | 49:01 | 01:02 | 13:02 |

| | | | | | |
|---|---|---|---|---|---|
| 10 | 02:01 | 13:02 | 39:24 | 07:01 | 13:03 |

| | | | | | | |
|---|---|---|---|---|---|---|
| 11 | 02:01 | 24:02 | 13:02 | 40:01 | 07:01 | 13:01 |
| 12 | 01:01 | 11:01 | 08:01 | 15:01 | 03:01 | 04:01 |

| | | | | | |
|---|---|---|---|---|---|
| 13 | 24:02 | 35:02 | 49:01 | 08:01 | 11:01 |
| 14 | 01:01 | 27:05 | 53:01 | 11:01 | 11:03 |

| | | | | | | |
|---|---|---|---|---|---|---|
| 15 | 01:01 | 68:02 | 08:01 | 51:01 | 03:01 | 16:01 |
| 16 | 02:01 | 03:01 | 44:02 | 49:01 | 11:01 | 15:01 |
| 17 | 03:01 | 26:01 | 41:01 | 55:01 | 10:01 | 14:01 |

T lymphocytes from the 17 donors, expanded in the presence of rENO1 for a week, were stimulated with irradiated autologous PBMCs as the antigen-presenting cells and loaded with the 14 ENO1 peptides in order to assess their proliferative capacity.

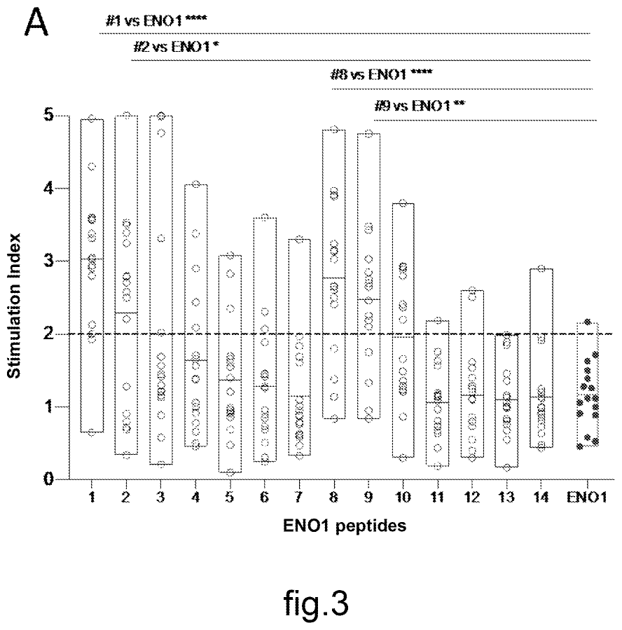

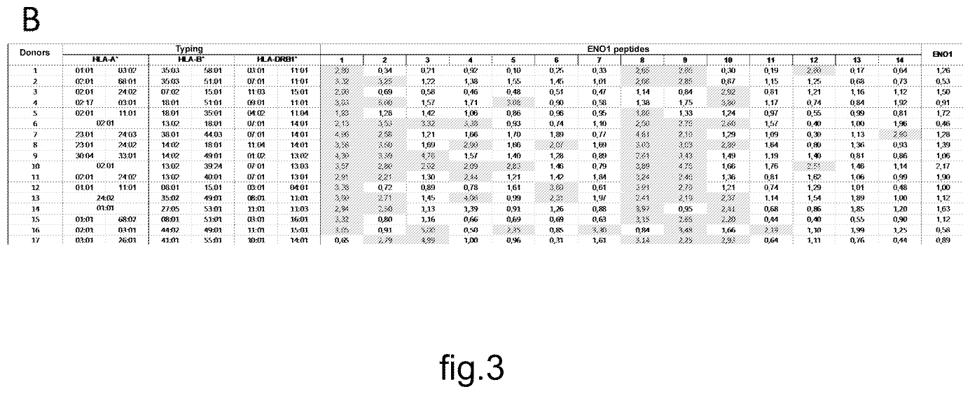

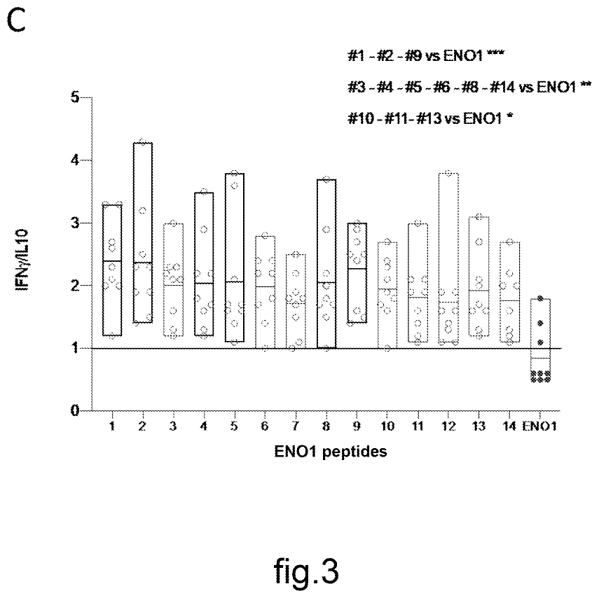

In general, the proliferative response to rENO1 only occurs in 1 out of 17 donors (6%) whereas, as shown in FIG. 3A-B, the peptides of SEQ ID NOs: 1 and 2 activate T lymphocyte proliferation (SI≥2) in 79% of donors, and the peptides of SEQ ID NOs: 8 and 9 activate proliferation in 82% of donors. Despite the high proliferative response to the peptides of SEQ ID NOs: 1, 2, 8 and 9, the in silico prediction showed that the HLA-A*02 gene, which is expressed in more than 25% of the Caucasian population, binds peptides 4 and 5 with higher affinity. In fact, as 4 out of 7 HLA-A*02 donors (57%) proliferate in response to the peptides of SEQ ID NOs: 4 and/or 5, these peptides were also selected.

In order to assess the immunological tone of the effector or suppressor response to stimulation with the individual peptides compared to rENO1, the ratio of IFN-γ to IL-10 production was measured (FIG. 3C). In general, the response induced by stimulation with rENO1 is predominantly suppressive compared to the effector response seen in T lymphocytes stimulated by all individual ENO1 peptides. Furthermore, peptides selected based on the proliferative response also show a significantly higher effector response than rENO1 (FIG. 3C).

### Validation of the ENO1 Immunogenic Peptides in a Cohort of PDAC Patients

ENO1 peptides (SEQ ID NOs: 1, 2, 4, 5, 8, and 9) selected by the previous in vitro assays were used to stimulate PBMCs of PDAC patients to test their proliferative index and immunological tone of the response.

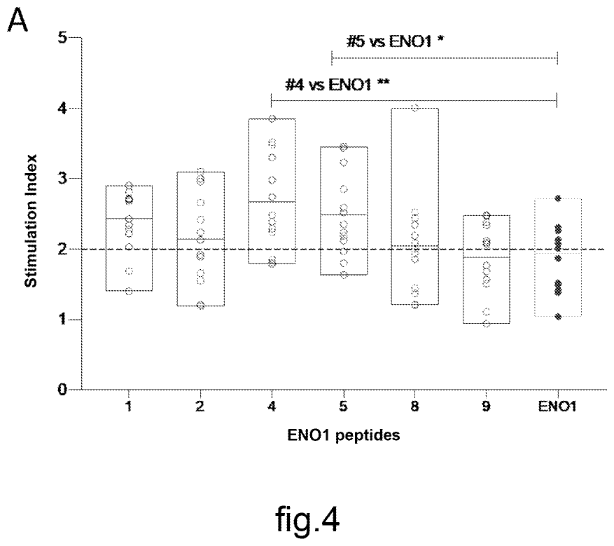

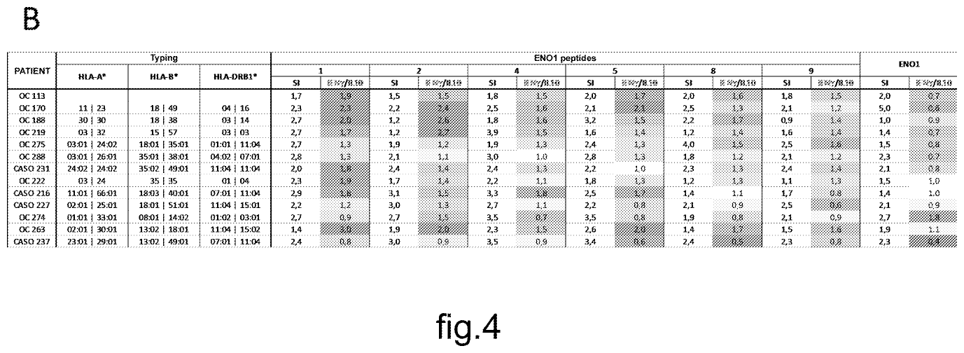

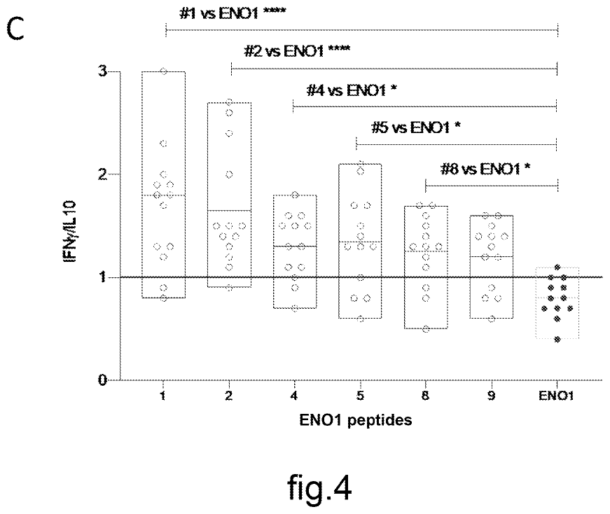

As shown in FIG. 4A-B, the proliferative response to rENO1 (SI>2) is observed in 7 out of 13 patients (53.8%), confirming previous studies on the expression of ENO1 in PDAC patients [Tomaino B, Cappello P, Capello M, et al. Circulating Autoantibodies to Phosphorylated a-Enolase Are a Hallmark of Pancreatic Cancer. J. Proteome Res. 2011;10(1):105-112], whereas stimulation with the selected peptides results in a proliferative response with SI>2 in 11 patients (84.6%). Analysis of the IFN-γ/IL-10 ratio shows that the immunological tone of the response to the individual peptides is fully oriented towards an effector response characterized by high IFN-γ production compared to the suppressive one observed by stimulating with rENO1 (FIG. 4B-C).

Table 3 provides the typing for the HLA-A, HLA-B and HLA-DRB1 loci, the SI, and the immunological tone for each patient. The cases in which, following stimulation with the selected peptides, the anti-tumor response improves, both in terms of proliferation and immunological tone, compared to rENO1 are highlighted in yellow. Following stimulation with rENO1, only 1 out of 13 patients (7.7%) exhibits an effector response and SI>2, whereas following stimulation with the ENO1 peptides, all patients (100%) exhibit an effector response and SI>2 (Table 3).

TABLE 3
Proliferative response and immunological tone in PDAC
patients stimulated with the selected peptides or rENO1

| | | | | [symbol] | | | | | | | | | | | | | |
|---|---|---|---|---|---|---|---|---|---|---|---|---|---|---|---|---|---|
| | [symbol] | | | 1 | | [symbol] | | 4 | | [symbol] | | 8 | | 9 | | [symbol] | |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | | | | 1.7 | 1.9 | 1.5 | 1.5 | 1.8 | 1.5 | 2.0 | 1.7 | 2.0 | [symbol] | 1.8 | 1.5 | 2.0 | 0.7 |
| [symbol] | [symbol] \| [symbol] | [symbol] \| [symbol] | [symbol] \| [symbol] | 2.3 | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] \| [symbol] | [symbol] \| [symbol] | [symbol] \| [symbol] | [symbol] | [symbol] | 1.2 | 2.5 | 1.8 | 1.6 | [symbol] | [symbol] | [symbol] | 1.7 | 0.9 | 1.4 | [symbol] | 0.9 |
| [symbol] | [symbol] \| [symbol] | [symbol] \| [symbol] | [symbol] \| [symbol] | [symbol] | 1.7 | [symbol] | 2.7 | [symbol] | [symbol] | 1.6 | 1.4 | [symbol] | [symbol] | [symbol] | [symbol] | [symbol] | 0.7 |
| [symbol] | [symbol] \| [symbol] | [symbol] \| [symbol] | [symbol] \| [symbol] | 2.7 | 1.3 | [symbol] | 1.2 | [symbol] | 1.3 | 2.4 | 1.3 | 4.0 | [symbol] | [symbol] | [symbol] | [symbol] | 0.8 |
| [symbol] | [symbol] \| [symbol] | [symbol] \| [symbol] | [symbol] \| [symbol] | 2.8 | [symbol] | 2.1 | [symbol] | [symbol] | [symbol] | [symbol] | [symbol] | [symbol] | [symbol] | 2.1 | [symbol] | [symbol] | 0.7 |
| [symbol] | [symbol] \| [symbol] | [symbol] \| [symbol] | [symbol] \| [symbol] | 2.0 | 1.8 | [symbol] | 1.4 | [symbol] | [symbol] | [symbol] | 1.0 | [symbol] | [symbol] | [symbol] | 1.4 | [symbol] | 0.8 |
| [symbol] | [symbol] \| [symbol] | [symbol] \| [symbol] | [symbol] \| [symbol] | [symbol] | 1.9 | 1.7 | 1.4 | 2.2 | 1.1 | 1.8 | 1.3 | 1.2 | [symbol] | 1.1 | 1.3 | [symbol] | 1.0 |
| [symbol] | [symbol] \| [symbol] | [symbol] \| [symbol] | [symbol] \| [symbol] | 2.9 | [symbol] | [symbol] | 1.5 | [symbol] | [symbol] | 2.5 | [symbol] | [symbol] | [symbol] | 1.7 | 0.8 | [symbol] | 1.6 |
| [symbol] | [symbol] \| [symbol] | [symbol] \| [symbol] | [symbol] \| [symbol] | 2.2 | 1.2 | 3.0 | 1.3 | [symbol] | [symbol] | [symbol] | 0.8 | 2.1 | [symbol] | 2.5 | 0.6 | 2.1 | 0.9 |
| [symbol] | [symbol] \| [symbol] | [symbol] \| [symbol] | [symbol] \| [symbol] | [symbol] | [symbol] | [symbol] | 1.5 | [symbol] | [symbol] | 3.5 | 0.8 | 1.9 | 0.8 | 2.1 | [symbol] | [symbol] | 1.8 |
| [symbol] | [symbol] \| [symbol] | [symbol] \| [symbol] | [symbol] \| [symbol] | 1.4 | 3.0 | [symbol] | 2.0 | [symbol] | [symbol] | 2.6 | [symbol] | [symbol] | 1.7 | [symbol] | [symbol] | [symbol] | 1.1 |
| [symbol] | [symbol] \| [symbol] | [symbol] \| [symbol] | [symbol] \| [symbol] | 2.4 | [symbol] | 3.0 | 0.9 | [symbol] | 0.9 | 3.4 | 0.6 | 2.4 | [symbol] | [symbol] | 0.8 | 2.3 | 0.4 |

[“PAZIENTE” = PATIENT; “Tipizzazione” = Typing; “Peptidi ENO1” = ENO1 peptides; “CASO” = CASE] [symbol]
indicates data missing or illegible when filed

The results in Table 3 indicate that the most immunogenic regions of ENO1 correspond to the sequences SEQ ID NOs: 1, 2, 4, 5, 8, and 9. These immunogenic sequences can be combined to obtain highly immunogenic synthetic peptides different from native human ENO1 fragments. Table 4 below shows some of these combinations (in addition to the amino acid sequence of each peptide, the table also shows the amino acid positions on the full-length sequence of ENO1 of the various regions that make up each peptide of the invention).

TABLE 4
Examples of immunogenic synthetic peptides of the invention

| SEQ | a.a. | |
|---|---|---|
| ID | positions | a.a. sequence |
| 15 | 1-80/ | MSILKIHAREIFDSRGNPTVEVDLFTSKGLFRAAVPSGASTGIYEALE |
| | 91-170/ | LRDNDKTRYMGKGVSKAVEHINKTIAPALVSKDKLMIEMDGTENK |
| | 211-290 | SKFGANAILGVSLAVCKAGAVEKGVPLYRHIADLAGNSEVILPVPAF |
| | | NVINGGSHAGNKLAMQEFMIGGFAPNILENKEGLELLKTAIGKAGY |
| | | TDKVVIGMDVAASEFFRSGKYDLDFKSPDDPSRYISPDQLADLYKSF |
| | | IKDYPVV |
| 16 | 1-80/ | MSILKIHAREIFDSRGNPTVEVDLFTSKGLFRAAVPSGASTGIYEALE |
| | 91-170 | LRDNDKTRYMGKGVSKAVEHINKTIAPALVSKDKLMIEMDGTENK |
| | | SKFGANAILGVSLAVCKAGAVEKGVPLYRHIADLAGNSEVILPVPAF |
| | | NVINGGSHAGNKLAMQEFMI |
| 17 | 1-80/ | MSILKIHAREIFDSRGNPTVEVDLFTSKGLFRAAVPSGASTGIYEALE |
| | 211-260 | LRDNDKTRYMGKGVSKAVEHINKTIAPALVSKGGFAPNILENKEGL |
| | | ELLKTAIGKAGYTDKVVIGMDVAASEFFRSGKYDLD |
| 18 | 91-170/ | DKLMIEMDGTENKSKFGANAILGVSLAVCKAGAVEKGVPLYRHIA |
| | 211-260 | DLAGNSEVILPVPAFNVINGGSHAGNKLAMQEFMIGGFAPNILENKE |
| | | GLELLKTAIGKAGYTDKVVIGMDVAASEFFRSGKYDLD |
| 19 | 1-50/ | MSILKIHAREIFDSRGNPTVEVDLFTSKGLFRAAVPSGASTGIYEALE |
| | 91-140/ | LRDKLMIEMDGTENKSKFGANAILGVSLAVCKAGAVEKGVPLYRHI |
| | 211-260 | ADLAGNGGFAPNILENKEGLELLKTAIGKAGYTDKVVIGMDVAASE |
| | | FFRSGKYDLD |
| 20 | 1-50/ | MSILKIHAREIFDSRGNPTVEVDLFTSKGLFRAAVPSGASTGIYEALE |
| | 121-170/ | LRAGAVEKGVPLYRHIADLAGNSEVILPVPAFNVINGGSHAGNKLA |
| | 211-260 | MQEFMIGGFAPNILENKEGLELLKTAIGKAGYTDKVVIGMDVAASE |
| | | FFRSGKYDLD |
| 21 | 1-80/ | MSILKIHAREIFDSRGNPTVEVDLFTSKGLFRAAVPSGASTGIYEALE |
| | 91-140 | LR |
| | | DNDKTRYMGKGVSKAVEHINKTIAPALVSKDKLMIEMDGTENKSK |
| | | FGANAILGVSLAVCKAGAVEKGVPLYRHIADLAGN |
| 22 | 1-80/ | MSILKIHAREIFDSRGNPTVEVDLFTSKGLFRAAVPSGASTGIYEALE |
| | 121-170 | LRDNDKTRYMGKGVSKAVEHINKTIAPALVSKAGAVEKGVPLYRHI |
| | | ADLAGNSEVILPVPAFNVINGGSHAGNKLAMQEFMI |
| 23 | 1-50/ | MSILKIHAREIFDSRGNPTVEVDLFTSKGLFRAAVPSGASTGIYEALE |
| | 91-170 | LRDKLMIEMDGTENKSKFGANAILGVSLAVCKAGAVEKGVPLYRHI |
| | | ADLAGNSEVILPVPAFNVINGGSHAGNKLAMQEFMI |
| 24 | 31-80/ | FRAAVPSGASTGIYEALELRDNDKTRYMGKGVSKAVEHINKTIAPA |
| | 91-170 | LVSKDKLMIEMDGTENKSKFGANAILGVSLAVCKAGAVEKGVPLY |
| | | RHIADLAGNAGAVEKGVPLYRHIADLAGNSEVILPVPAFNVINGGSH |
| | | AGNKLAMQEFMI |
| 25 | 31-80/ | FRAAVPSGASTGIYEALELRDNDKTRYMGKGVSKAVEHINKTIAPA |
| | 91-140/ | LVSKDKLMIEMDGTENKSKFGANAILGVSLAVCKAGAVEKGVPLY |
| | 211-260 | RHIADLAGNGGFAPNILENKEGLELLKTAIGKAGYTDKVVIGMDVA |
| | | ASEFFRSGKYDLD |
| 26 | 31-80/ | FRAAVPSGASTGIYEALELRDNDKTRYMGKGVSKAVEHINKTIAPA |
| | 121-170/ | LVSKAGAVEKGVPLYRHIADLAGNSEVILPVPAFNVINGGSHAGNK |
| | 211-260 | LAMQEFMIGGFAPNILENKEGLELLKTAIGKAGYTDKVVIGMDVAA |
| | | SEFFRSGKYDLD |
| 27 | 121-170/ | AGAVEKGVPLYRHIADLAGNSEVILPVPAFNVINGGSHAGNKLAMQ |
| | 211-260 | EFMIGGFAPNILENKEGLELLKTAIGKAGYTDKVVIGMDVAASEFFR |
| | | SGKYDLD |
| 28 | 1-50/ | MSILKIHAREIFDSRGNPTVEVDLFTSKGLFRAAVPSGASTGIYEALE |
| | 91-140 | LRDKLMIEMDGTENKSKFGANAILGVSLAVCKAGAVEKGVPLYRHI |
| | | ADLAGN |
| 29 | 1-50/ | MSILKIHAREIFDSRGNPTVEVDLFTSKGLFRAAVPSGASTGIYEALE |
| | 121-170 | LRAGAVEKGVPLYRHIADLAGNSEVILPVPAFNVINGGSHAGNKLA |
| | | MQEFMI |
| 30 | 1-50/ | MSILKIHAREIFDSRGNPTVEVDLFTSKGLFRAAVPSGASTGIYEALE |
| | 211-260 | LRGGFAPNILENKEGLELLKTAIGKAGYTDKVVIGMDVAASEFFRSG |
| | | KYDLD |
| 31 | 31-80/ | FRAAVPSGASTGIYEALELRDNDKTRYMGKGVSKAVEHINKTIAPA |
| | 91-140 | LVSKDKLMIEMDGTENKSKFGANAILGVSLAVCKAGAVEKGVPLY |
| | | RHIADLAGN |
| 32 | 31-80/ | FRAAVPSGASTGIYEALELRDNDKTRYMGKGVSKAVEHINKTIAPA |
| | 121-170 | LVSKAGAVEKGVPLYRHIADLAGNSEVILPVPAFNVINGGSHAGNK |
| | | LAMQEFMI |
| 33 | 31-80/ | FRAAVPSGASTGIYEALELRDNDKTRYMGKGVSKAVEHINKTIAPA |
| | 211-260 | LVSKGGFAPNILENKEGLELLKTAIGKAGYTDKVVIGMDVAASEFFR |
| | | SGKYDLD |
| 34 | 91-140/ | DKLMIEMDGTENKSKFGANAILGVSLAVCKAGAVEKGVPLYRHIA |
| | 211-260 | DLAGNGGFAPNILENKEGLELLKTAIGKAGYTDKVVIGMDVAASEF |
| | | FRSGKYDLD |
| 35 | 1-80/ | MSILKIHAREIFDSRGNPTVEVDLFTSKGLFRAAVPSGASTGIYEALE |
| | 121-170/ | LRDNDKTRYMGKGVSKAVEHINKTIAPALVSKAGAVEKGVPLYRHI |
| | 211-260 | ADLAGNSEVILPVPAFNVINGGSHAGNKLAMQEFMIGGFAPNILENK |
| | | EGLELLKTAIGKAGYTDKVVIGMDVAASEFFRSGKYDLD |
| 36 | 1-80/ | MSILKIHAREIFDSRGNPTVEVDLFTSKGLFRAAVPSGASTGIYEALE |
| | 91-140/ | LRDNDKTRYMGKGVSKAVEHINKTIAPALVSKDKLMIEMDGTENK |
| | 211-260 | SKFGANAILGVSLAVCKAGAVEKGVPLYRHIADLAGNGGFAPNILE |
| | | NKEGLELLKTAIGKAGYTDKVVIGMDVAASEFFRSGKYDLD |
| 37 | 31-80/ | FRAAVPSGASTGIYEALELRDNDKTRYMGKGVSKAVEHINKTIAPA |
| | 91-170/ | LVSKDKLMIEMDGTENKSKFGANAILGVSLAVCKAGAVEKGVPLY |
| | 211-260 | RHIADLAGNSEVILPVPAFNVINGGSHAGNKLAMQEFMIGGFAPNIL |
| | | ENKEGLELLKTAIGKAGYTDKVVIGMDVAASEFFRSGKYDLD |
| 38 | 1-50/ | MSILKIHAREIFDSRGNPTVEVDLFTSKGLFRAAVPSGASTGIYEALE |
| | 91-170/ | LRDKLMIEMDGTENKSKFGANAILGVSLAVCKAGAVEKGVPLYRHI |
| | 211-260 | ADLAGNSEVILPVPAFNVINGGSHAGNKLAMQEFMIGGFAPNILENK |
| | | EGLELLKTAIGKAGYTDKVVIGMDVAASEFFRSGKYDLD |

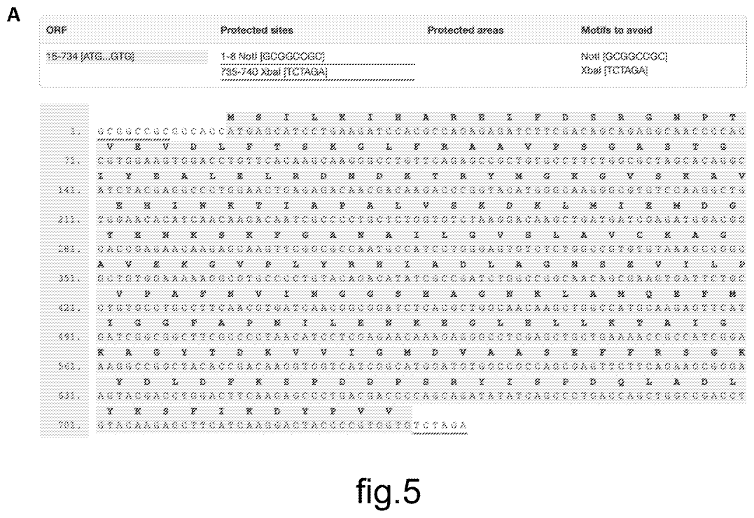

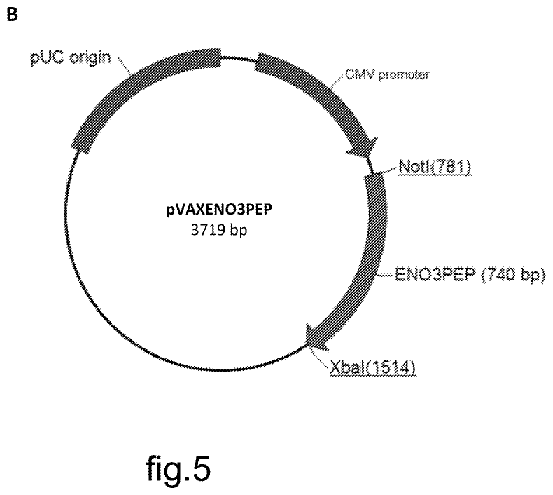

The preferred combination is SEQ ID NO:15. In a preferred embodiment, the sequence coding for SEQ ID NO:15 is preceded by a single initial Kozak sequence so that no new epitopes are created (FIG. 5A). The construct features an origin of replication site (pUC), an antibiotic-resistance site to select only the bacteria that have successfully integrated the sequence, a CMV promoter that allows replication, and the two restriction sites for the enzymes NotI and XbaI to allow cDNA insertion. The map of the vector with these features, already approved for clinical use, is shown in FIG. 5B.

### Validation of the In Vivo Therapeutic Potential of pVAXENO3PEP

Mice genetically engineered (GEM) to spontaneously develop PDAC were vaccinated either with the empty pVAX plasmid or with the pVAX plasmid encoding SEQ ID 15 (pVAXENO3PEP) or full-length ENO1 (pVAXENO1) following the previously described protocol [Cappello P, Rolla S, Chiarle R, et al. Vaccination with ENO1 DNA prolongs survival of genetically engineered mice with pancreatic cancer. Gastroenterology. 2013;144(5):1098-1106]. One month after the last vaccination, the animals were sacrificed to test for: (i) the size of the tumor area, (ii) the titer of ENO1-specific antibodies, (iii) the number of T lymphocytes secreting IFN-γ in response to ENO1, (iv) the immune infiltrate in the tumor area.

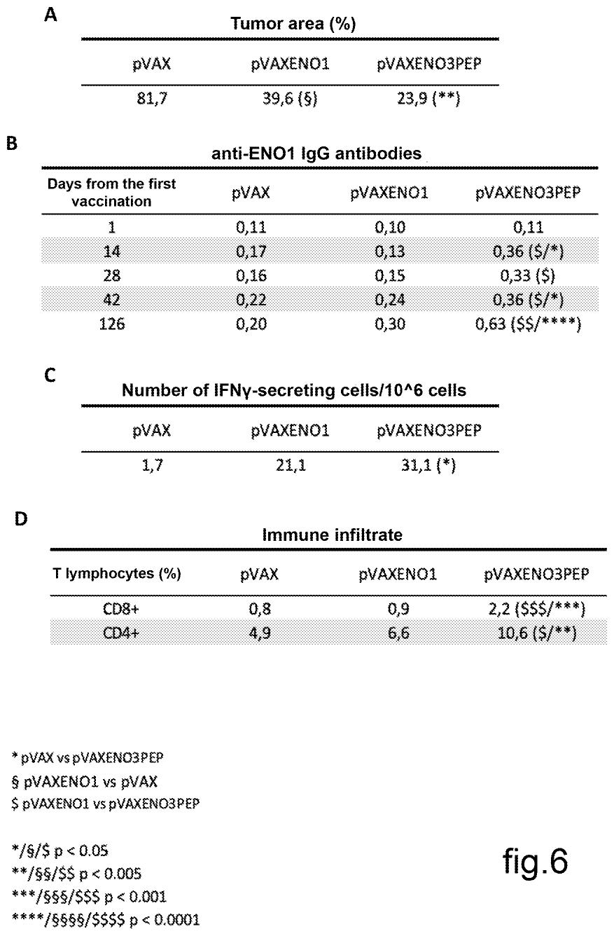

Analysis of the tumor area showed a significantly greater reduction in tumor lesions in mice vaccinated with pVAXENO3PEP than in control mice (FIG. 6A). The reduction in the tumor area induced by vaccination with pVAXENO3PEP is accompanied by an early increase and higher titer of anti-ENO1 antibodies (FIG. 6B) as well as an increased number of IFN-γ-secreting T lymphocytes (FIG. 6C).

Activation of T lymphocytes is also shown by an increased presence of CD4+ and CD8+ T lymphocytes in the tumor area (FIG. 6D).

### References

1. Siegel R L, Miller K D, Jemal A. Cancer Statistics, 2020. CA. Cancer J. Clin. 2020;70(1):7-30
2. Tomaino B, Cappello P, Capello M, et al. Circulating Autoantibodies to Phosphorylated a-Enolase Are a Hallmark of Pancreatic Cancer. J. Proteome Res. 2011;10(1):105-112.
3. Cappello P, Tomaino B, Chiarle R, et al. An Integrated Humoral and Cellular Response Is Elicited in Pancreatic Cancer by a-Enolase, a Novel Pancreatic Ductal Adenocarcinoma-Associated Antigen. Int. J. Cancer. 2009; 125(3):639-648.
4. Amedei A, Niccolai E, Benagiano M, Della Bella C. et al. Ex Vivo Analysis of Pancreatic Cancer-Infiltrating T Lymphocytes Reveals That ENO-Specific Tregs Accumulate in Tumor Tissue and Inhibit Th1/Th17 Effector Cell Functions. Cancer Immunol. Immunother. 2013;62(7):1249-1260.
5. WO2011/030302 A1: An isolated monophosphorylated peptide derived from human alpha-enolase useful for diagnosis and treatment of pancreatic adenocarcinoma, antibodies directed against the said monophosphorylated peptide, and uses thereof. Novelli F, Tomaino B, Cappello P.
6. Huang CK, Sun Y, Lv L, et al. ENO1 and Cancer. Molecular therapy oncolytics, 2022;24:288-298.
7. Cappello P, Principe M, Bulfamante S, et al. Front Biosci (Landmark Ed). 2017;22(5):944-959.
8. Almaguel F A, Sanchez T W, Ortiz-Hernandez et al. Front Genet. 2021; 11:614726
9. WO 2007/072219: ALPHA ENOLASE-DIRECTED DIAGNOSTICS AND THERAPEUTICS FOR CANCER AND CHEMOTHERAPEUTIC DRUG RESISTANCE. Georges E, Prinos P.
10. WO 2016/170139: Novel peptides and combination of peptides for use in immunotherapy against lung cancer, including NSCLC and other cancers. Mahr A, Weinschenk T, Schoor O, Fritsche J, Singh H, Wagner C, Leibold J, Song C.
11. Cappello P, Rolla S, Chiarle R, et al. Vaccination with ENO1 DNA prolongs survival of genetically engineered mice with pancreatic cancer. Gastroenterology. 2013; 144(5): 1098-1106.
12. Capello M, Caorsi C, Bogantes Hernadez P J, et al. Phosphorylated alpha-enolase induces autoantibodies in HLA-DR8 pancreatic cancer patients and triggers HLA-DR8 restricted T cell activation. Immunology Letters. 2015;167(1):11-16.
13. WO2017/013425: ANTI-TUMOUR IMMUNE RESPONSES TO MODIFIED SELF-EPITOPES. Durrant L G, Brentville V A, Metheringham R L.
14. Kinloch A, Tatzer V, Wait R et al. Identification of citrullinated alpha-enolase as a candidate autoantigen in rheumatoid arthritis. Arthritis Res Ther. 2005;7(6):R1421-9.
15. Lundberg K, Kinloch A, Fisher B A, et al. Autoantibodies to citrullinated alpha-enolase peptide 1 are specific rheumatoid arthritis and cross-react with bacterial enolase. Arthritis Reum. 2008;58(10):3009-19.
16. Mahdi H, Fisher BA, Kallberg H et al. Specific interaction between genotype, smoking and autoimmunity to citrullinated alpha-enolase in the etiology of rheumatoid arthritis. Nat Genet. 2009;41(12):1319-24
17. Amoroso A, Ferrero N M, Rendine S. Le Caratteristiche HLA Della Popolazione Italiana: Analisi Di 370.000 Volontari Iscritti All'IBMDR. Analysis 2010, 1-2, 23-102.

What is claimed is

1-21. (canceled)

22. A recombinant expression vector comprising a recombinant nucleotide sequence coding for an immunogenic synthetic peptide of SEQ ID NO:15, said recombinant nucleotide sequence being operatively linked to a promoter sequence and optionally to additional transcription regulatory elements.

23. The recombinant expression vector of claim 22, wherein said recombinant nucleotide sequence comprises a polyadenylation signal.

24. The recombinant expression vector of claim 22, wherein the recombinant expression vector is unable to replicate in a mammalian cell.

25. The recombinant expression vector of claim 22, wherein the recombinant expression vector is a plasmid vector.

26. An immunogenic synthetic peptide of SEQ ID NO: 15.

27. An isolated nucleic acid coding for the immunogenic synthetic peptide of claim 26.

28. A pharmaceutical composition comprising the recombinant expression vector of claim 22 or the immunogenic synthetic peptide of SEQ ID NO: 15, in combination with at least one pharmaceutically acceptable carrier, excipient, diluent, stabilizer and/or preservative.

29. The pharmaceutical composition of claim 28, wherein the recombinant expression vector is adsorbed on polylactide-co-glycolide (PLG) microparticles.

30. The pharmaceutical composition of claim 28, comprising an adjuvant selected from the group consisting of toll-like receptor agonists, high-mobility group protein B1 (HMGB1), INKT lymphocyte synthetic agonists, and γδ T lymphocyte agonists.

31. The pharmaceutical composition of claim 28, wherein the pharmaceutical composition is in a form suitable for oral, nasal, subcutaneous, intradermal, or intramuscular administration.

32. A method for eliciting an immune response a subject having a tumor, the method comprising administering to the subject the pharmaceutical composition of claim 28.

33. The method of claim 32, wherein the tumor is pancreatic ductal adenocarcinoma.

34. The method of claim 32, wherein the subject is a human or an animal.

35. The method of claim 32, wherein the subject is a mammal.

36. A method for eliciting an immune response a subject having a tumor, the method comprising administering to the subject a combined preparation comprising the recombinant expression vector of claim 22 or the immunogenic synthetic peptide of SEQ ID NO: 15 and at least one chemotherapeutic agent and/or at least one immuno-modulating agent.

37. The method of claim 36, wherein the tumor is pancreatic ductal adenocarcinoma.


SEQUENCE LISTING (40 sequences)

SEQ ID NO: 1 - protein, 50 aa, Homo sapiens
MSILKIHAREIFDSRGNPTVEVDLFTSKGLFRAAVPSGASTGIYEALELR

SEQ ID NO: 2 - protein, 50 aa, Homo sapiens
FRAAVPSGASTGIYEALELRDNDKTRYMGKGVSKAVEHINKTIAPALVSK

SEQ ID NO: 3 - protein, 50 aa, Homo sapiens
GVSKAVEHINKTIAPALVSKKLNVTEQEKIDKLMIEMDGTENKSKFGANA

SEQ ID NO: 4 - protein, 50 aa, Homo sapiens
DKLMIEMDGTENKSKFGANAILGVSLAVCKAGAVEKGVPLYRHIADLAGN

SEQ ID NO: 5 - protein, 50 aa, Homo sapiens
AGAVEKGVPLYRHIADLAGNSEVILPVPAFNVINGGSHAGNKLAMQEFMI

SEQ ID NO: 6 - protein, 50 aa, Homo sapiens
NVINGGSHAGNKLAMQEFMILPVGAANFREAMRIGAEVYHNLKNVIKEKY

SEQ ID NO: 7 - protein, 50 aa, Homo sapiens
AMRIGAEVYHNLKNVIKEKYGKDATNVGDEGGFAPNILENKEGLELLKTA

SEQ ID NO: 8 - protein, 50 aa, Homo sapiens
GGFAPNILENKEGLELLKTAIGKAGYTDKVVIGMDVAASEFFRSGKYDLD

SEQ ID NO: 9 - protein, 50 aa, Homo sapiens
VIGMDVAASEFFRSGKYDLDFKSPDDPSRYISPDQLADLYKSFIKDYPVV

SEQ ID NO: 10 - protein, 50 aa, Homo sapiens
ISPDQLADLYKSFIKDYPVVSIEDPFDQDDWGAWQKFTASAGIQVVGDDL

SEQ ID NO: 11 - protein, 50 aa, Homo sapiens
WGAWQKFTASAGIQVVGDDLTVTNPKRIAKAVNEKSCNCLLLKVNQIGSV

SEQ ID NO: 12 - protein, 50 aa, Homo sapiens
AVNEKSCNCLLLKVNQIGSVTESLQACKLAQANGWGVMVSHRSGETEDTF

SEQ ID NO: 13 - protein, 50 aa, Homo sapiens
QANGWGVMVSHRSGETEDTFIADLVVGLCTGQIKTGAPCRSERLAKYNQL

SEQ ID NO: 14 - protein, 50 aa, Homo sapiens; identical to SEQ ID NO: 13

SEQ ID NO: 15 - protein, 240 aa, synthetic construct
MSILKIHAREIFDSRGNPTVEVDLFTSKGLFRAAVPSGASTGIYEALELRDNDKTRYMGKGVSKAVEHINKTIAPALVSKDKLMIEMDGTENKSKFGANAILGVSLAVCKAGAVEKGVPLYRHIADLAGNSEVILPVPAFNVINGGSHAGNKLAMQEFMIGGFAPNILENKEGLELLKTAIGKAGYTDKVVIGMDVAASEFFRSGKYDLDFKSPDDPSRYISPDQLADLYKSFIKDYPVV

SEQ ID NO: 16 - protein, 160 aa, synthetic construct
MSILKIHAREIFDSRGNPTVEVDLFTSKGLFRAAVPSGASTGIYEALELRDNDKTRYMGKGVSKAVEHINKTIAPALVSKDKLMIEMDGTENKSKFGANAILGVSLAVCKAGAVEKGVPLYRHIADLAGNSEVILPVPAFNVINGGSHAGNKLAMQEFMI

SEQ ID NO: 17 - protein, 130 aa, synthetic construct
MSILKIHAREIFDSRGNPTVEVDLFTSKGLFRAAVPSGASTGIYEALELRDNDKTRYMGKGVSKAVEHINKTIAPALVSKGGFAPNILENKEGLELLKTAIGKAGYTDKVVIGMDVAASEFFRSGKYDLD

SEQ ID NO: 18 - protein, 130 aa, synthetic construct
DKLMIEMDGTENKSKFGANAILGVSLAVCKAGAVEKGVPLYRHIADLAGNSEVILPVPAFNVINGGSHAGNKLAMQEFMIGGFAPNILENKEGLELLKTAIGKAGYTDKVVIGMDVAASEFFRSGKYDLD

SEQ ID NO: 19 - protein, 150 aa, synthetic construct
MSILKIHAREIFDSRGNPTVEVDLFTSKGLFRAAVPSGASTGIYEALELRDKLMIEMDGTENKSKFGANAILGVSLAVCKAGAVEKGVPLYRHIADLAGNGGFAPNILENKEGLELLKTAIGKAGYTDKVVIGMDVAASEFFRSGKYDLD

SEQ ID NO: 20 - protein, 150 aa, synthetic construct
MSILKIHAREIFDSRGNPTVEVDLFTSKGLFRAAVPSGASTGIYEALELRAGAVEKGVPLYRHIADLAGNSEVILPVPAFNVINGGSHAGNKLAMQEFMIGGFAPNILENKEGLELLKTAIGKAGYTDKVVIGMDVAASEFFRSGKYDLD

SEQ ID NO: 21 - protein, 130 aa, synthetic construct
MSILKIHAREIFDSRGNPTVEVDLFTSKGLFRAAVPSGASTGIYEALELRDNDKTRYMGKGVSKAVEHINKTIAPALVSKDKLMIEMDGTENKSKFGANAILGVSLAVCKAGAVEKGVPLYRHIADLAGN

SEQ ID NO: 22 - protein, 130 aa, synthetic construct
MSILKIHAREIFDSRGNPTVEVDLFTSKGLFRAAVPSGASTGIYEALELRDNDKTRYMGKGVSKAVEHINKTIAPALVSKAGAVEKGVPLYRHIADLAGNSEVILPVPAFNVINGGSHAGNKLAMQEFMI

SEQ ID NO: 23 - protein, 130 aa, synthetic construct
MSILKIHAREIFDSRGNPTVEVDLFTSKGLFRAAVPSGASTGIYEALELRDKLMIEMDGTENKSKFGANAILGVSLAVCKAGAVEKGVPLYRHIADLAGNSEVILPVPAFNVINGGSHAGNKLAMQEFMI

SEQ ID NO: 24 - protein, 150 aa, synthetic construct
FRAAVPSGASTGIYEALELRDNDKTRYMGKGVSKAVEHINKTIAPALVSKDKLMIEMDGTENKSKFGANAILGVSLAVCKAGAVEKGVPLYRHIADLAGNAGAVEKGVPLYRHIADLAGNSEVILPVPAFNVINGGSHAGNKLAMQEFMI

SEQ ID NO: 25 - protein, 150 aa, synthetic construct
FRAAVPSGASTGIYEALELRDNDKTRYMGKGVSKAVEHINKTIAPALVSKDKLMIEMDGTENKSKFGANAILGVSLAVCKAGAVEKGVPLYRHIADLAGNGGFAPNILENKEGLELLKTAIGKAGYTDKVVIGMDVAASEFFRSGKYDLD

SEQ ID NO: 26 - protein, 150 aa, synthetic construct
FRAAVPSGASTGIYEALELRDNDKTRYMGKGVSKAVEHINKTIAPALVSKAGAVEKGVPLYRHIADLAGNSEVILPVPAFNVINGGSHAGNKLAMQEFMIGGFAPNILENKEGLELLKTAIGKAGYTDKVVIGMDVAASEFFRSGKYDLD

SEQ ID NO: 27 - protein, 100 aa, synthetic construct
AGAVEKGVPLYRHIADLAGNSEVILPVPAFNVINGGSHAGNKLAMQEFMIGGFAPNILENKEGLELLKTAIGKAGYTDKVVIGMDVAASEFFRSGKYDLD

SEQ ID NO: 28 - protein, 100 aa, synthetic construct
MSILKIHAREIFDSRGNPTVEVDLFTSKGLFRAAVPSGASTGIYEALELRDKLMIEMDGTENKSKFGANAILGVSLAVCKAGAVEKGVPLYRHIADLAGN

SEQ ID NO: 29 - protein, 100 aa, synthetic construct
MSILKIHAREIFDSRGNPTVEVDLFTSKGLFRAAVPSGASTGIYEALELRAGAVEKGVPLYRHIADLAGNSEVILPVPAFNVINGGSHAGNKLAMQEFMI

SEQ ID NO: 30 - protein, 100 aa, synthetic construct
MSILKIHAREIFDSRGNPTVEVDLFTSKGLFRAAVPSGASTGIYEALELRGGFAPNILENKEGLELLKTAIGKAGYTDKVVIGMDVAASEFFRSGKYDLD

SEQ ID NO: 31 - protein, 100 aa, synthetic construct
FRAAVPSGASTGIYEALELRDNDKTRYMGKGVSKAVEHINKTIAPALVSKDKLMIEMDGTENKSKFGANAILGVSLAVCKAGAVEKGVPLYRHIADLAGN

SEQ ID NO: 32 - protein, 100 aa, synthetic construct
FRAAVPSGASTGIYEALELRDNDKTRYMGKGVSKAVEHINKTIAPALVSKAGAVEKGVPLYRHIADLAGNSEVILPVPAFNVINGGSHAGNKLAMQEFMI

SEQ ID NO: 33 - protein, 100 aa, synthetic construct
FRAAVPSGASTGIYEALELRDNDKTRYMGKGVSKAVEHINKTIAPALVSKGGFAPNILENKEGLELLKTAIGKAGYTDKVVIGMDVAASEFFRSGKYDLD

SEQ ID NO: 34 - protein, 100 aa, synthetic construct
DKLMIEMDGTENKSKFGANAILGVSLAVCKAGAVEKGVPLYRHIADLAGNGGFAPNILENKEGLELLKTAIGKAGYTDKVVIGMDVAASEFFRSGKYDLD

SEQ ID NO: 35 - protein, 180 aa, synthetic construct
MSILKIHAREIFDSRGNPTVEVDLFTSKGLFRAAVPSGASTGIYEALELRDNDKTRYMGKGVSKAVEHINKTIAPALVSKAGAVEKGVPLYRHIADLAGNSEVILPVPAFNVINGGSHAGNKLAMQEFMIGGFAPNILENKEGLELLKTAIGKAGYTDKVVIGMDVAASEFFRSGKYDLD

SEQ ID NO: 36 - protein, 180 aa, synthetic construct
MSILKIHAREIFDSRGNPTVEVDLFTSKGLFRAAVPSGASTGIYEALELRDNDKTRYMGKGVSKAVEHINKTIAPALVSKDKLMIEMDGTENKSKFGANAILGVSLAVCKAGAVEKGVPLYRHIADLAGNGGFAPNILENKEGLELLKTAIGKAGYTDKVVIGMDVAASEFFRSGKYDLD

SEQ ID NO: 37 - protein, 180 aa, synthetic construct
FRAAVPSGASTGIYEALELRDNDKTRYMGKGVSKAVEHINKTIAPALVSKDKLMIEMDGTENKSKFGANAILGVSLAVCKAGAVEKGVPLYRHIADLAGNSEVILPVPAFNVINGGSHAGNKLAMQEFMIGGFAPNILENKEGLELLKTAIGKAGYTDKVVIGMDVAASEFFRSGKYDLD

SEQ ID NO: 38 - protein, 180 aa, synthetic construct
MSILKIHAREIFDSRGNPTVEVDLFTSKGLFRAAVPSGASTGIYEALELRDKLMIEMDGTENKSKFGANAILGVSLAVCKAGAVEKGVPLYRHIADLAGNSEVILPVPAFNVINGGSHAGNKLAMQEFMIGGFAPNILENKEGLELLKTAIGKAGYTDKVVIGMDVAASEFFRSGKYDLD

SEQ ID NO: 39 - protein, 434 aa, synthetic construct
MSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 40 - DNA, 740 bp, synthetic construct
  source 1..740: mol_type = other DNA
gcggccgcgccaccatgagcatcctgaagatccacgccagagagatcttcgacagcagaggcaaccccaccgtggaagtggacctgttcacaagcaagggcctgttcagagccgctgtgccttctggcgctagcacaggcatctacgaggccctggaactgagagacaacgacaagacccggtacatgggcaagggcgtgtccaaggctgtggaacacatcaacaagacaatcgcccctgctctggtgtctaaggacaagctgatgatcgagatggacggcaccgagaacaagagcaagttcggcgccaatgccatcctgggagtgtctctggccgtgtgtaaagccggcgctgtggaaaaaggcgtgcccctgtacagacatatcgccgatctggccggcaacagcgaagtgattctgcctgtgcctgccttcaacgtgatcaacggcggatctcacgctggcaacaagctggcaatgcaagagttcatgatcggcggcttcgcccctaacatcctcgagaacaaagagggcctcgagctgctgaaaaccgccatcggaaaggccggctacaccgacaaggtggtcatcggcatggatgtggccgccagcgagttcttcagaagcgggaagtacgacctggacttcaagagccctgagcaccccagcagatatatcagccctgaccagctggccgacctgtacaagagcttcatcaaggactaccccgtggtgtctaga